(19) United States
(12) Patent Application Publication
(10) Pub. No.: US 2003/0077238 A1
(43) Pub. Date: Apr. 24, 2003

(54) Cosmetic kit and method

(76) Inventors: Maria Magdalena Catharina Roovers, Roosendaal (NL); Rudi Westerkamp, Roosendaal (NL); Pierre Blais, Ottawa (CA)

Correspondence Address: Robert S. Lipton, Esquire, LIPTON WEINBERGER & HUSICK, 201 N. Jackson Street, P.O. Box 934, Media, PA, 19063-0934, US

(21) Appl. No.: 10/236,658

(22) Filed: Sep. 6, 2002

Related U.S. Application Data
(60) Provisional application No. 60/318,093, filed on Sep. 7, 2001.

(51) Int. Cl. A61K 7/021; A61K 33/08; A61K 33/00

(52) U.S. Cl. 424/63; 424/195.18; 424/724; 424/691

(57) ABSTRACT

The invention is a cosmetic incorporating the concepts of Traditional Chinese Medicine. Powdered gemstones and pigments selected for their therapeutic effect are blended into a cosmetic powder. A consumer applies a skin gel to the user's skin, lips or hair. The consumer applies the cosmetic powder to the skin gel. Powdered gemstones and pigments may be added to nail coating for application to nails. The consumer realizes the benefits of the gemstones under Traditional Chinese Medicine through proximity of the gemstones to the consumer when used as a cosmetic.

### PRIORITY CLAIM

This application claims the benefit of U.S. Provisional Application No., 60/318,093 filed on Sept. 7, 2001.

### BACKGROUND OF THE INVENTION

1. Field of the Invention

The invention relates generally to the field of cosmetic preparations and specifically to cosmetic preparations incorporating concepts of Traditional Chinese Medicine (‘TCM’). The Invention is a kit comprising powdered gemstones reputed to have salutary effects under TCM that are blended into a pigmented powder. For the purposes of this application, the term “gemstones” means one or more of amethyst, rose quartz, ruby, carnelian, aventurine, aquamarine, jade and amber. For purposes of the application, “supporting gemstones” means one or more of diamond, sapphire, emerald, mountain crystal and freshwater pearls. The kit of the Invention also comprises a skin gel to fix the pigmented powder in place. The skin gel may be premixed with the pigmented powder or may be separate from the pigmented powder. For nails, the kit may include lacquer or other nail coating. The invention further comprises a method for administering TCM through application of the cosmetic of the Invention.

2. Description of Related Art

Introduction: To a Western observer, Chinese Traditional Medicine may seem one of the more foreign aspects of a very foreign culture. Whether or not one believes in the teachings of Traditional Chinese Medicine (‘TCM’), it is undeniable that many people around the world firmly do believe. To those people and perhaps to non-believers as well, the beneficial effects of TCM are very real. Whether a beneficial effect is the product of qi (discussed below) or the placebo effect is not relevant. What is important is that the consumer experiences the beneficial effect for whatever reason. The present invention incorporates into modern cosmetics certain gemstones for the purpose of securing the beneficial effect of those gemstones to the consumer using the principals of TCM.

TCM has a very long history of efficacy in Asia. At least one practice of TCM, acupuncture, has gained wide acceptance in Western culture. Acupuncture is based on the same principles of TCM expressed below.

General Concepts of Traditional Chinese Medicine: The chief principle upon which TCM is based is that life and health result from the proper flow of qi (pronounced ‘chee’), or energy, through an extensive network of channels in the body. Unlike western medicine, TCM concerns itself primarily with the flow of qi rather than chemistry and matter. Health, and by analogy beauty, can be seen as a function of a person's energy, rather than as a function of their skin and bones.

Yin Yang Theory: Yin-Yang theory is based on the philosophical construct of two polar complements, called Yin and Yang. These complementary opposites are neither forces nor material entities. They are convenient labels used to describe how things, function in relation to each other and to the universe. They are used to explain the continuous process of natural change. But Yin and Yang represent a way of thinking, where all things are seen as parts of a whole. No entity can ever be isolated from its relationship to other entities; no thing can exist in and of itself.

The character for Yin originally meant the shady side of a slope. It is associated with such qualities as cold, rest, responsiveness, passivity, darkness, interiority, downwardness, inwardness, and decrease. The original meaning of Yang was the sunny side of a slope. The term implies brightness and is part of one common Chinese expression for the sun. Yang is associated with qualities such as heat, stimulation, movement, activity, excitement, vigour, light, exteriority, upward-ness, outward-ness, and increase. Working with these ideas, Chinese thought and Chinese medical tradition developed sophisticated principles of Yin and Yang.

Three Treasures (Sanbao): The Three Treasures of TCM are the three energies of jing, qi and shen. In the physical world they are analogous to jing for the solid phase, qi for the liquid phase and shen for the gaseous phase.

Jing is best translated as essence, is the substance that underlies all organic life. It is the source of organic change. Generally thought of as fluid like, jing is supportive and nutritive, and is the basis of reproduction and development. Jing has two sources, which are also its characteristic aspects. Prenatal jing (xian-tian--zhi--jing), also translated as Congenital Essence, is inherited from the parents. In fact, the fusion of this parental jing is conception. Each person's prenatal jing is unique and will determine his or her particular growth patterns. The quantity and quality of the prenatal jing is fixed at birth and, together with original qi, determines an individual's basic makeup and constitution. Postnatal jing (hou-tian-zhi-jing) is the second source and aspect of jing. It is derived from the purified parts of ingested food. The postnatal jing constantly adds vitality to the prenatal jing. Together, they comprise the overall jing of the body.

Qi is fundamental to Chinese medical thinking, yet no one English word or phrase can adequately capture its meaning. According to TCM theory, everything in the universe, organic and inorganic, is composed of and defined by its qi. But qi is not some primordial, immutable material, nor is it merely vital energy, although the word is occasionally so translated. Chinese thought does not distinguish between matter and energy, but we can perhaps think of qi as matter on the verge of becoming energy, or energy at the point of materializing. Traditional Chinese medicine is based on the concept that a person is composed of qi or bio-energy. The Asian idea of health is the proper balance of energy in a person. This is unlike the concepts of western medicine which has up until recent times held the concept that health is the absence of destructive organisms, (bacteria, viruses, etc.) and the proper balance of bio-chemicals in the body. It is important for a proper understanding of our cosmetics to start seeing life as a flow of energy and to understand that each person is composed of energy that can be felt as emotion. There are two kinds of qi in a person. The first is nourishing qi, which is yin and flows inside the body through channels called meridians. The second is defensive qi, which is yang and surrounds a person's body like a protective aura.

Shen is best translated as Spirit. It is an elusive concept, perhaps because, in the medical tradition, it is the Substance unique to human life. If jing is the source of life, and qi the ability to activate and move, then shen is the vitality behind jing and qi in the human body. While animate and inanimate movement are indicative of qi, and instinctual organic processes reflect jing, human consciousness indicates the presence of shen. Shen is associated with the force of human personality, the ability to think, discriminate, and choose appropriately, or, as is commonly said: “Shen is the awareness that shines out of our eyes when we are truly awake.” The origin of shen is analogous to the origin of jing: Each parent contributes to the creation of the offspring's shen, yet the shen is also continuously and materially nourished after birth. Although the English word Spirit may be used to translate shen, shen does have a material aspect. It is a Fundamental Substance of the human body and has no importance to medicine independent of the body.

The Five Phases (Wu Xing): The theory of the Five Phases is an attempt to classify phenomena in terms of five quintessential processes, represented by the symbols of Wood, Fire, Earth, Metal, and Water. We know that energy is never lost, and the theory of the Five Phases is that it merely transforms itself through a cycle of these phases. The Five Phases are not in any way ultimate constituents of matter. The Chinese term that we translate as “Five Phases” is wu xing. Wu is the number five, and xing means “walk” or “move,” and perhaps most pertinently, it implies a process. The wu xing, therefore, are five kinds of processes; hence the Five Phases, and not the Five Elements. The theory of Phases is a system of correspondences and patterns that subsume events and things, especially in relationship to their dynamics.

More specifically, each Phase is an emblem that denotes a category of related functions and qualities. The Phase called Wood is associated with active functions that are in a growing phase. Fire designates functions that have reached a maximal state of activity and are about to begin a decline or a resting period. Metal represents functions in a declining state. Water represents functions that have reached a maximal state of rest and are about to change the direction of their activity. Finally, Earth designates balance or neutrality; in a sense, Earth is a buffer between the other Phases. In the sense, that the Phases correlate observable phenomena of human life into images derived from the macrocosm, they serve a similar function as that of elements in other medical systems.

In more concrete terms, the Five Phases can be used to describe the annual cycle in terms of biological growth and development. Wood corresponds to spring, Fire to summer, Metal to autumn, and Water to winter. Earth may represent the transition between each season (and it is commonly used to represent “Indian summer”). These correlations are known as the Mutual Production order of the Five Phases. They represent the way in which the Five Phases interact and arise out of one another in the typical yearly cycle. There are thirty-six mathematically possible orders in which the Five Phases can be arranged, but only a few of them are actually used either in medicine or in other disciplines.

Eight Diagrams (Bagua): According to the principles of TCM, the energy that animates the world, qi, can be split into eight different sub-energies, in the same way that white light can be split into the colors of the rainbow. These eight sub-energies are called the bagua. These eight energies are the basic ingredients of everything material, and immaterial, visible and invisible. The eight energies are symbolized by eight diagrams. Each diagram is a binary code of three horizontal lines, broken and unbroken. There are called Heaven, Lake, Fire, Thunder, Wind, Water, Mountain and Earth.

### SUMMARY OF THE INVENTION

The present Invention is the combination of gemstones having salutary effects under concepts of Traditional Chinese Medicine (“TCM”) into modern cosmetic products for personal use, particularly into powders. Gemstones or gemstones and pigments are selected using principals of TCM so as to achieve desired therapeutic effects. The gemstones and pigments are reduced to a pigmented powder. The skin gel is applied to the skin, lips, hair or for any other cosmetic application, and the pigmented powder is applied to the skin gel. For nails, a lacquer or any other conventional nail coating is mixed with the pigmented powder and applied to the nails. The consumer realizes the benefits of TCM through having the gemstones and pigments incorporated into the cosmetic in contact with or in close proximity to the consumers' body. The pigmented powder may be applied to nails using lacquer or other conventional nail treatments. The pigmented powder may be mixed with the gel during manufacture and prior to application for convenience of the consumer. The pigmented powder may be applied to the body, such as to acupuncture points, as a powder and without a skin gel or other adhesive.

It is an object of the present invention to provide a cosmetic composition that is based on a synthesis of Traditional Chinese Medicine, Traditional Gemstone Lore and modern science.

It is an object of the present Invention to provide a cosmetic composition that nourishes the qi in the body of the wearer, firstly by nourishing the qi that flows in the meridians under the skin and at important acupuncture sites (this is a form of inner beauty), and secondly in nourishing the defensive qi that is like the aura around the wearer (this is a form of outer beauty).

It is an object of the present Invention to provide a cosmetic composition that utilizes the TCM concepts of Yin & Yang, the theory of the Three Treasures (sanbao), the concept of the Eight Diagrams (bagua), and the concept of the Five Phases (wu xing).

It is an object of the present Invention to provide a cosmetic composition that utilizes Traditional Gemstone Lore attributing beneficial properties to gemstones and minerals when worn against the skin.

It is an object of the present Invention to provide a cosmetic product that is multi-functional, i.e. it can be used on different parts of the body, e.g. the lips, the areas around the eyes, the nails and other areas of skin.

### BRIEF DESCRIPTION OF THE DRAWINGS

FIG. 1 is a flow diagram of the method of the Invention.

FIG. 2 is a flow diagram using a skin gel.

FIG. 3 is a flow diagram using a nail coating.

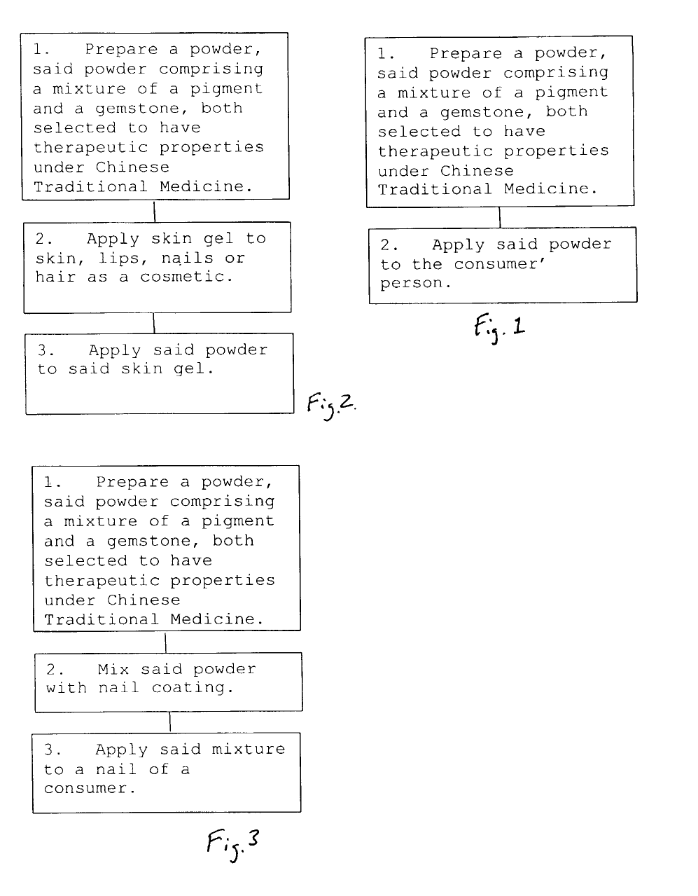

### DESCRIPTION OF AN EMBODIMENT

The present Invention comprises two main components:

1. A series of pigmented powders is created comprising pigments and gemstones. The series of pigmented powders is divided into groups by color corresponding to the eight bagua energies (heaven, lake, fire, thunder, wind, water, mountain and earth) and eight different effects on the skin, as well as two powders of varying shades of black and white divided into yin and yang.

2. A long-lasting, preservative free, skin gel that may include synthetic compositions, ultra violet filters and vitamins is created as a make-up base, to bind the pigmented powder to the skin or hair.

As more fully described below, the consumer applies the skin gel to his or her skin, lips or hair. The consumer then selects a pigmented powder and applies the pigmented powder to the skin gel. The pigmented powder, including the powdered gemstones, adheres to the skin gel and hence to the consumer's skin, lips or hair. The consumer selects the powder for aesthetic effect and to secure the benefits of the pigmented powder under concepts of TCM.

The consumer also may apply the pigmented powder without the skin gel or may apply the pigmented powder to nails without the skin gel and in combination with a lacquer or other conventional nail coating.

Selection of Gemstones

Gemstones are divided by color into two categories: gemstones having colors that purify (Yin) and gemstones having colors containing that nourish (Yang). Each color and gem was chosen to enhance the power and effect. For example, cool colors have a natural, purifying influence. Hot colors and gems, like red and rose quartz, have a nourishing effect. Gemstones also are divided into the eight energies of the bagua. The division is as follows:

| | |
|---|---|
| Amethyst for heaven energy | Yang |
| Rose quartz for lake energy | Yang |
| Ruby for fire energy | Yang |
| Carnelian for thunder energy | Yang |
| Aventurine for wind energy | Yin |
| Aquamarine for water energy | Yin |
| Jade for mountain energy | Yin |
| Amber for earth energy | Yin |

Bagua and Traditional Gemstone Lore attributes emotional and personality characteristics to the gemstones as follows:

| | | | | |
|---|---|---|---|---|
| Amethyst | = | Heaven | = | Creativity, Inspiration & Easy |
| | | | | Activeness |
| Rose quartz | = | Lake | = | Enthusiasm, Joy & Youthfulness |
| Ruby | = | Fire | = | Passion, Determination & Vitality |
| Carnelian | = | Thunder | = | Erotic Feelings & Empowerment |
| Aventurine | = | Wind | = | Thoughtfulness, Gentleness & |
| | | | | Softness |
| Aquamarine | = | Water | = | Relaxation, Purifying & Cooling |
| Jade | = | Mountain | = | Satisfaction, Contentment & |
| | | | | Harmony, Calmness |
| Amber | = | Earth | = | Nurturing & Caring |

From Traditional Gemstone Lore, the gemstones have beneficial properties when worn against the skin. The properties of the gemstones are:

| | |
|---|---|
| Amethyst: | Relaxes the skin |
| Rose quartz: | Rejuvenates the skin, renews the cells |
| Ruby: | Nourishes the skin |
| Carnelian: | Revitalizes the skin, stimulates blood circulation to keep |
| | the skin vital |
| Amber: | Bestows the healing power of the earth, makes the skin |
| | pleasurable and soft |
| Jade: | Harmonizes the skin and keeps it beautiful |
| Aquamarine: | Known as the “skin-care stone”, removes skin irritation |
| Blue | Promotes the health of the skin |
| Aventurine: | |
| Sapphire: | Promotes the shen energy |
| Emerald: | Promotes the qi energy |
| Diamond: | Promotes the jing energy |
| Mountain | Promotes nourishing qi energy |
| Crystal: | |
| Mica: | Protects the skin from negative elements in the |
| | environment. |

According to the theory of the Three Treasures (sanbao) in Traditional Chinese Medicine and Traditional Gemstone Lore, gemstones will nourishing the three life energies of jing, qi and shen when applied to the body areas corresponding to these three energies, i.e. the nails & hair, the lips and the areas around the eyes. Also according to the theory of the Three Treasures (sanbao) in TCM and Traditional Gemstone Lore, diamond will intensify jing energy, emerald will intensify qi energy and sapphire will intensify shen energy in conjunction with other of the gemstones.

Chemical Composition of Pigmented Powders:

A pigmented powder base is formulated comprising conventional pigments. Eight different blends of pigmented powder are created corresponding with each of the eight bagua energies. The pigmented powders consist of:

| | Percentage By |
|---|---|
| Ingredient | Weight |
| Conventional color pigments and pigment base, which | 90-99 |
| may include pearlized pigments containing | |
| mica and titanium dioxide | |

Up to four of the following powdered gemstones or minerals are used in each colored powder composition:

| | | | |
|---|---|---|---|
| 1. | Amber | (Rosin) | 1-3 |
| 2. | Amethyst | (Quartz) | 1-3 |
| 3. | Aquamarine | (Beryl) | 1-3 |
| 4. | Blue Aventurine | (Quartz) | 1-3 |
| 5. | Carnelian | (Quartz) | 1-3 |
| 6. | Diamond | (Carbon) | 1-3 |
| 7. | Emerald | (Beryl) | 1-3 |
| 8. | Jade | (Nephrite-Actinolite) | 1-3 |
| 9. | Mountain Crystal | (Quartz) | 1-3 |
| 10. | Rose Quartz | (Quartz) | 1-3 |
| 11. | Ruby | (Corundum) | 1-3 |
| 12. | Sapphire | (Corundum) | 1-3 |
| 13. | Natural fresh water pearl | (calcium carbonate) | 1-3 |

Note:
The gemstones and minerals have a particle size of approximately one micron.

Ingredients of the eight energy (bagua) powders:

(a) Heaven energy color powders:

purple pigment

powdered amethyst

and one or more of

powdered sapphire

powdered diamond

powdered emerald

powdered natural fresh water pearls

(b) Lake energy color powders:

pink pigments

powdered rose quartz

and one or more of

powdered sapphire

powdered diamond

powdered emerald

powdered natural fresh water pearls

(c) Fire energy color powders:

red pigments

powdered ruby

and one or more of

powdered sapphire

powdered diamond

powdered emerald

powdered natural fresh water pearls

(d) Thunder energy color powders:

bronze pigments

powdered carnelian

and one or more of

powdered sapphire

powdered diamond

powdered emerald

powdered natural fresh water pearls

(e) Wind energy color powders:

light blue pigments

powdered Aventurine

and one or more of

powdered sapphire

powdered diamond

powdered emerald

powdered natural fresh water pearls

(f) Water energy color powders:

blue pigments

powdered aquamarine

and one or more of

powdered sapphire

powdered diamond

powdered emerald

powdered natural fresh water pearls

(g) Mountain energy color powders:

green pigments

powdered jade

and one or more of

powdered sapphire

powdered diamond

powdered emerald

powdered natural fresh water pearls

(h) Earth energy color powders:

gold pigments

powdered amber

and one or more of

powdered sapphire

powdered diamond

powdered emerald

powdered natural fresh water pearls

(i) Yin energy black colored powders

black pigments

and one or more of

powdered amethyst

powdered mountain crystal

powdered rose quartz

powdered ruby

powdered carnelian

(j) Yang energy white colored powders

white pigment

and one or more of

powdered adventurine

powdered mountain crystal

powdered aquamarine

powdered jade

powdered amber

Method of Manufacture of the Pigmented Powder

The following steps are followed to produce the pigmented powder of the present Invention.

1. Gemstones are selected to achieve a desired therapeutic result using the principles of TCM described above.

2. The gemstones are ground to a fine powder. The optimal particle size of the powder is one micron.

3. The gemstones are then washed and sterilized by heat or by any other suitable method.

4. The gemstones are mechanically mixed with the pigment base in the proper proportions.

5. The resulting mixture is packaged as a loose powder in individual sterile containers for the consumer.

Chemical Composition of Skin Gel

The following formulae are examples for graded skin gels with a range of properties suitable for widely differing skin properties. All are derived from the same ingredients where the percentage by weight of key components are adjusted to achieve the desired properties.

TABLE #1
Composition range for gels suited for dry skin

| | | Percentage by |
|---|---|---|
| Ingredient | Description | Weight |
| Polymer Base | Poly glyceryl methacrylate | 6.0-12.0 |
| | Poly glyceryl acrylate | 0.0-6.0 |
| | Hydroxypropylcellulose | 1.0-3.0 |
| | Polydimethylsiloxane | 1.0-4.0 |
| Solvent Base | Cyclosimethicone | 10.0-20.0 |
| | Propylene glycol | 20.0-50.0 |
| | Glycerol | 2.0-7.0 |
| | Ethyl alchohol | 1.0-3.0 |
| | Water (deionized) | 5.0-10.0 |
| Functional Additives | Vitamin E | 0.1-0.5 |
| | Vitamin C | 0.1-0.5 |
| | Octyl dodecyl meadowfoamate | 1.0-2.0 |
| | Methyl gluceth 10 | 1.0-3.0 |
| Pigment Additives* | Polytetrafluorethylene | less than 0.001 |
| | Perfluorodecalin | less than 0.001 |
| | FD & C Blue #1 | less than 0.001 |
| | Iron oxides | less than 0.001 |
| | Aluminum oxide | less than 0.001 |
| | Titanium dioxide | less than 0.001 |

*optional additives without functional role other than to impart a pale coloration which is not transferred to skin after application of the product

TABLE #2
Composition range for gels suited for oily skin

| | | Percentage by |
|---|---|---|
| Ingredient | Description | Weight |
| Polymer Base | Poly glyceryl methacrylate | 6.0-12.0 |
| | Poly glyceryl acrylate | 0.0-6.0 |
| | Hydroxypropylcellulose | 0.0-1.0 |
| | Polydimethylsiloxane | 0.5-1.0 |
| Solvent Base | Cyclosimethicone | 5.0-15.0 |
| | Propylene glycol | 10.0-40.0 |
| | Glycerol | 0.0-3.0 |
| | Ethyl alchohol | 2.0-4.0 |
| | Water (deionized) | 7.0-15.0 |
| Functional Additives | Vitamin E | 0.0-0.5 |
| | Vitamin C | 0.1-0.5 |
| | Octyl dodecyl meadowfoamate | 0.0-1.0 |
| | Methyl gluceth 10 | 0.0-1.0 |
| Pigment Additives* | Polytetrafluorethylene | less than 0.001 |
| | Perfluorodecalin | less than 0.001 |
| | FD & C Blue #1 | less than 0.001 |
| | Iron oxides | less than 0.001 |
| | Aluminum oxide | less than 0.001 |
| | Titanium dioxide | less than 0.001 |

*optional additives without functional role other than to impart a pale coloration which is not transferred to skin after application of the product

Composition range of gels suited for intermediate skin characteristics

The composition ranges stated in ingredient Table #1 (dry skin) and Table #2 (oily skin) provided above constitute the extremes for each gel preparation. Compositions suited for “normal” skin characteristics are interpolated from the ingredient Tables #1 and #2. Production of such gels are simplified through the blending of a single master batch of which sub-batches are made for each skin category by adding supplemental components or increasing the amount of selected components in accord with the ingredient Tables #1 and #2. The formulae components responsible for “dryness” or “oiliness” are readjusted to offset the undesirable property of the user's skin. For example, in the case of a user with an objectionably dry skin, components such as glycerol, polydimethysiloxane, vitamin E and octyl dodecyl meadowfoamate are increased to compensate for the lack of moisture retention of the user's skin. Conversely, gels intended for users with oily skin have reduced oil-bases components and increased components which absorb or disperse natural oils. These changes reduce the excess of oils at the skin's surface. Components such as hydroxypropylcellulose, ethyl alchohol and methyl gluceth 10 are increased and the oily components such as vitamin E and octyl dodecyl meadowfoamate are reduced.

Advantages of the described approach to formulation include commonality of components for sub-types of products and mutual compatibility of the components for each sub-type. This allows production of larger quantities and blending of master-batches. It also allows the use of simple formulae, computer software and nomographs to produced batches of user-specific products. Economic advantages arise from purchases of larger quantities of intermediates, simpler blending methods and pre-compounding of sub-components, for example, glyceryl methacrylate and glyceryl acrylate pre-compounded with propylene glycol, cylcosimethicone and glycerol.

A gels made according to the preceding formulae generally is dermatologically secure, inert, clean and sterile. The gel is preservative free, contains no added fragrance and is colorless and odorless on the skin. The gel helps to protect the skin from environmental damage and helps the skin retain moisture.

A gel made according to the preceding formula will adhere to the skin, and pigmented powder will adhere to the gel. The gel can fix pigmented powder for eye shadow, can act as a base for lipstick, or can act as a base for make-up and foundation powder. The gel may bind the pigmented powder to the skin for an extended period of time. Vitamins and emollients may be included in the gel to promote skin health.

Method of Manufacture of the Skin Gel

Manufacturing of batches on direct blending at room temperature in stirred vessels is acceptable, as opposed to complex stage-wise preparation with heat and blending. A process for manufacturing is described below.

The preparation of principal ingredients for immediate use or for storage is performed first. Master-batches of the polymer base can be obtained by blending solids with selected solvents at ambient temperature using a high viscosity blending apparatus preferably at low shear. Concentrations up to 30% by weight of poly glyceryl methacrylate and/or poly acrylate with either propylene glycol, glycerol and/or cyclosimethicone can be obtained. Alternatively, these products can be procured as prepared intermediates available as market commodities or subcontracted from suppliers of cosmetic intermediates. Concentrates of the additives can be prepared similarly at room temperature using conventional blending equipment for low viscosity products, such as propeller or impeller type closed vessel systems.

From each of the purchased or specially prepared concentrates, calculated mixtures from the previously-cited formulae and guidelines, are then blended by adding supplemental solvent(s) and additive(s) unique to the desired batch. Calculations can be simplified through the use of numerical control methods, nomographs or computer software developed from input data derived from the composition ranges set forth in the ingredient Tables #1 and #2 provided above. Technologies and computer programs suitable for calculations are commercially available or can be derived from existing standardized spreadsheet software.

Method of Use of the Invention:

For skin and hair:

1. The consumer can use their fingers, a sponge, a brush or another applicator.

2. The applicator can be used dry, dampened with water, or moistened with some of the gel.

3. The gel can also be first applied directly to the area of the skin or hair to which the pigmented powder is to be applied.

4. The consumer lightly touches their fingers or the applicator into to the container of the desired pigmented powder, and applies it to the area of the skin or hair to which the color is desired.

For nails:

For nail color, the consumer dips a clear nail lacquer into the pigmented powder of choice and applies the resulting mixture directly onto the nails. Further coatings can be applied to give a heavier coat. For purposes of this application, the term “nail coating” means lacquer or any of the many conventional means for applying a coating to nails.

Acupuncture sites:

The consumer can also apply the color powder to acupuncture site on the face and body to especially nourish the qi that flows in these sites.

The combination of the gel base and colored powder is mulitfunctional, i.e. it can be used on different parts of the body, e.g. the hair, the lips, the areas around the eyes, and other areas of skin. For the purposes of this application, a ‘consumer's person’ means any part of the body of a consumer, including without limitation skin, lips, nails and hair.

Many different embodiments of the above invention are possible. This application is intended to address all possible embodiments and is limited only as described in the following claims.

I claim:

1. A cosmetic kit comprising:
a. a plurality of gemstones selected so as to have desired salutary properties under concepts of Traditional Chinese Medicine; said gemstones being reduced to a powder.
b. a pigment reduced to a powder and mixed with said gemstone powder to form a powdered mixture.

2. The cosmetic kit of claim 1 further comprising said gemstone being selected from the group consisting of amethyst, rose quartz, ruby, carnelian, aventurine, aquamarine, jade and amber.

3. The cosmetic kit of claim 1, further comprising a plurality of supporting gemstones, said plurality of supporting gemstones being selected from the list consisting of sapphire, diamond, emerald and natural freshwater pearls; said plurality of supporting gemstones being reduced to a powder and mixed with said powdered mixture.

4. The cosmetic kit of claim 3, said plurality of gemstones comprising amethyst, said pigment comprising a purple pigment.

5. The cosmetic kit of claim 3, said plurality of gemstones comprising rose quartz, said pigment comprising a pink pigment.

6. The cosmetic kit of claim 3, said plurality of gemstones comprising ruby, said pigment comprising a red pigment.

7. The cosmetic kit of claim 3, said plurality of gemstones comprising carnelian, said pigment comprising a bronze pigment.

8. The cosmetic kit of claim 3, said plurality of gemstones comprising averturine, said pigment comprising a light blue pigment.

9. The cosmetic kit of claim 3, said plurality of gemstones comprising aquamarine, said pigment comprising a blue pigment.

10. The cosmetic kit of claim 3, said plurality of gemstones comprising jade, said pigment comprising a green pigment.

11. The cosmetic kit of claim 3, said plurality of gemstones comprising amber, said pigment comprising a gold pigment.

12. The cosmetic kit of claim 3, said pigments comprising 90-99% of said powdered mixture; said gemstone comprising 1-3% of said powdered mixture; and each of said supporting gemstones present in said plurality of supporting gemstones comprising 1-3% of said powdered mixture, where all percentages are by weight and where a “-” represents a range of allowable percentages.

13. The cosmetic kit of claim 12, further comprising a nail coating, said nail coating adapted to be mixed with said powdered mixture and applied to a nail of a consumer and further adapted to hold said powdered mixture in place on said nail of said consumer.

14. The cosmetic kit of claim 12, further comprising a skin gel, said skin gel adapted to hold said powdered mixture in place on a consumer's skin or hair.

15. The cosmetic kit of claim 14, said skin gel being mixed with said powdered mixture.

16. The cosmetic kit of claim 14, said skin gel comprising a polymer base, a solvent base, functional additives and pigment additives.

17. The cosmetic kit of claim 16, said polymer base consisting of Poly glyceryl methacrylate at 6.0-12.0%, Poly glyceryl acrylate at 0.0-6.0%, Hydroxypropylcellulose at 1.0-3.0% by weight, Polydimethylsiloxane at 1.0-4.0%; said solvent base consisting of closimethicone at 10.0-20.0%, Propylene glycol at 20.0-50.0%, Glycerol at 2.0-7.0%, Ethyl alchohol at 1.0-3.0%, Water (deionized) at 5.0-10.0%; said functional additives consisting of vitamin E at 0.1-0.5%, Vitamin C at 0.1-0.5%, Octyl dodecyl meadowfoamate at 1.0-2.0%, and Methyl gluceth 10 at 1.0-3.0%; and said pigment additives consisting of Polytetrafluorethylene at less than 0.001%, Perfluorodecalin at less than 0.001%, FD & C Blue #1 at less than 0.001%, Iron oxides at less than 0.001%, Aluminum oxide at less than 0.001%, and Titanium dioxide at less than 0.001%, where all percentages are by weight and where a “-” represents a range of allowable percentages.

18. The cosmetic kit of claim 16, said polymer base consisting of Poly glyceryl methacrylate at 6.0-12.0%, Poly glyceryl acrylate at 0.0-6.0%, Hydroxypropylcellulose at 0-1.0%, Polydimethylsiloxane at 0.5-1.0%; said solvent base consisting of closimethicone at 5.0-15.0%, Propylene glycol at 10.0-40.0%, Glycerol at 0-3.0%, Ethyl alchohol at 2.0-4.0%, Water (deionized) at 7.0-15.0%; said functional additives consisting of vitamin E at 0-0.5%, Vitamin C at 0.1-0.5%, Octyl dodecyl meadowfoamate at 0-1.0%, and Methyl gluceth 10 at 0-1.0%; and said pigment additives consisting of Polytetrafluorethylene at less than 0.001%, Perfluorodecalin at less than 0.001%, FD & C Blue #1 at less than 0.001%, Iron oxides at less than 0.001%, Aluminum oxide at less than 0.001%, and Titanium dioxide at less than 0.001%, where all percentages are by weight and where a “-” represents a range of allowable percentages.

19. The cosmetic kit of claim 16, said polymer base consisting of Poly glyceryl methacrylate at 6.0-12.0%, Poly glyceryl acrylate at 0.0-6.0%, Hydroxypropylcellulose at 0-3.0% by weight, Polydimethylsiloxane at 0.5-4.0%; said solvent base consisting of closimethicone at 5.0-20.0%, Propylene glycol at 10.0-50.0%, Glycerol at 0-7.0%, Ethyl alchohol at 1.0-4.0%, Water (deionized) at 5.0-15.0%; said functional additives consisting of vitamin E at 0-0.5%, Vitamin C at 0.1 - 0.5%, Octyl dodecyl meadowfoamate at 0-2.0%, and Methyl gluceth 10 at 0-3.0%; and said pigment additives consisting of Polytetrafluorethylene at 0-0.001%, Perfluorodecalin at 0-0.001%, FD & C Blue #1 at 0-0.001%, Iron oxides at 0-0.001%, Aluminum oxide at 0-0.001%, and Titanium dioxide at 0-0.001%, where all percentages are by weight and where a “-” represents a range of allowable percentages.

20. The cosmetic kit of claim 1 further comprising said gemstone being selected from the group consisting of amethyst, mountain crystal, rose quartz, ruby and carnelian and said pigment being a black pigment.

21. The cosmetic kit of claim 1 further comprising said gemstone being selected from the group consisting of aventurine, mountain crystal, aquamarine, jade and amber and said pigment being a white pigment.

22. A method of administering Traditional Chinese Medicine in a cosmetic comprising:
a. preparing a powder comprising a mixture of a pigment and a gemstone, said gemstone and said pigment being selected so as to have desired therapeutic properties under concepts of Traditional Chinese Medicine; and
b. applying said powder to a consumer's person.

23. The method of claim 22, said gemstone being selected from the list consisting of amethyst, rose quartz, ruby, carnelian, aventurine, aquamarine, jade, and amber.

24. The method of claim 23, said powder further comprising a plurality of powdered supporting gemstones, said plurality of supporting gemstones being selected from the list consisting of sapphire, diamond, emerald and natural freshwater pearls.

25. The method of claim 24, said pigments comprising 90-99% of said powder; said gemstone comprising 1-3% of said powder; and each of said supporting gemstones in said plurality of supporting gemstones comprising 1-3% of said powder, where all percentages are by weight and where a “-” represents a range of allowable percentages.

26. The method of claim 25, said method including the additional step of applying a skin gel to the consumer's person prior to said step of applying said powder to said consumer's person, said skin gel being adapted to adhere said powder to said skin or hair of said consumer, said skin gel comprising a polymer base, a solvent base, functional additives and pigment additives.

27. The method of claim 25, said step of applying said powder to said consumer's person comprising the steps of mixing said powder with a nail coating, and applying said nail coating to a nail of said consumer.

28. The method of claim 25 comprising the additional step of mixing said powder with a skin gel prior to the step of applying said powder to said consumer's person, said skin gel being adapted to adhere said powder to said skin or hair of said consumer, said skin gel comprising a polymer base, a solvent base, functional additives and pigment additives.
